(19) United States
(12) Patent Application Publication
(10) Pub. No.: US 2024/0327775 A1
(43) Pub. Date: Oct. 3, 2024

(54) TRAINING METHOD AND DEVICE FOR MACHINE LEARNING MODEL

(71) Applicant: HITACHI HIGH-TECH CORPORATION, Tokyo (JP)

(72) Inventors: Mitsuji IKEDA, Tokyo (JP); Makoto KATAGISHI, Tokyo (JP); Yuichi ABE, Tokyo (JP); Yohei MINEKAWA, Tokyo (JP)

(21) Appl. No.: 18/580,132

(22) PCT Filed: Sep. 27, 2021
(86) PCT No.: PCT/JP2021/035420

§ 371 Date: Jan. 17, 2024

(51) Int. Cl. C12M 1/36

(52) U.S. Cl. CPC C12M 41/48

(57) ABSTRACT

A training method for a machine learning model can be trained for predicting characteristics after cell culture. The training method for machine learning models includes first performing a cell culture experiment at least three times, in which a plurality of cell images are captured during or after cell culture, and after the cell culture, a plurality of pieces of characteristic information of cells of the plurality of cell images are acquired, and second using some or all of pairs combining the plurality of captured cell images and the characteristic information as training data. In the second step, information indicating a magnitude relationship of the plurality of pieces of characteristic information of the plurality of cell images is classified into at least three classes including large, medium and small classes, and some of the cell images of the large and small classes are trained as the training data.

### TECHNICAL FIELD

The present invention relates to a training method and device for a machine learning model for the purpose of predicting the characteristics of cells after cell culture.

### BACKGROUND ART

For example, pluripotent stem cells are stem cells that can differentiate into most types of cells, and are expected to be applied to drug discovery, regenerative medicine, and the like. However, the technology to culture pluripotent stem cells and efficiently induce differentiation into specific cells or cell tissues is still at the research level, and the establishment of effective technology is awaited.

It is necessary to confirm whether the cultured or differentiated cells meet the expected characteristics to establish an effective technology.

Generally, evaluation of expression using undifferentiated markers and differentiation markers, and evaluation of cells after differentiation induction using fluorescence microscopy and flow cytometry, and the like are performed.

There is also a technology using machine learning, as shown in PTL 1.

### CITATION LIST

### Patent Literature

PTL 1: JP2021-43600A

### SUMMARY OF INVENTION

### Technical Problem

The technology described in PTL 1 is a method for generating a learning model that induces differentiation of artificial pluripotent stem cells into predetermined cells, acquires cell information of the cells induced to differentiate into the predetermined cells, acquires training data including the cell information and physical property information of the cell based on measurement by an analyzer, generates a learning model that outputs the physical property information of the cell based on the training data in response to input of the cell information of the cell induced to differentiate into the predetermined cell.

The cell information includes cell images after culture, and the like. The physical property information includes cell viability, and the like.

Regarding the technology described in PTL 1, the cell information is merely a plurality of unstained cell images, and when the physical property information is specific numerical information such as cell viability, although not described in PTL 1, an approach can be contemplated in which a plurality of datasets with the same labels corresponding to numerical information for all cell images are prepared and trained. It is believed that such an approach will enable concrete and appropriate training.

In other words, if the physical property (characteristic) is specific numerical information, it is possible to perform specific training and perform more accurate training if a plurality of datasets with the same label corresponding to numerical information can be prepared for all cell images and used for training.

However, the approach described above has a problem that many incorrectly labeled cell images are generated, and correct training cannot be performed.

It is thought that in the image from the culture experiment in which the physical properties are at an intermediate level, information that has a positive effect on the physical properties and information that has a negative effect on the physical properties are equally mixed. Therefore, when the same labels are attached, it is thought that there are many images with labels that do not match the information included in the images, that is, there are many incorrectly labeled images. The incorrectly labeled images make training difficult.

To solve the problems described above, an object of the present invention is to provide a training method and device for a machine learning model that can be trained more appropriately for predicting characteristics after cell culture.

### Solution to Problem

To achieve the object described above, the present invention is configured as follows.

A training method for a machine learning model includes a first step of performing a cell culture experiment at least three times, in which a plurality of cell images are captured during or after the cell culture, and after the cell culture, a plurality of pieces of characteristic information of cells of the plurality of cell images are acquired, and a second step of using some or all of pairs combining the plurality of captured cell images and the characteristic information as training data, in which the second step classifies information indicating a magnitude relationship of the plurality of pieces of characteristic information of the plurality of cell images into at least three classes including large, medium and small classes, and trains the cell images of the large and small classes as the training data.

A training device includes a first data storage unit that stores a plurality of cell images captured during or after cell culture and characteristic information of cells of the plurality of cell images, a data selection unit that classifies information indicating a magnitude relationship of a plurality of pieces of the characteristic information stored in the first data storage unit into at least three classes including large, medium and small classes, and selects the cell images of the large and small classes, a second data storage unit that stores the cell images of the large and small classes selected by the data selection unit together with the characteristic information, and a machine learning model that is trained by using, as training data, the cell images of the large and small classes stored in the second data storage unit.

### Advantageous Effects of Invention

It is possible to provide a training method and device for a machine learning model that can be trained more appropriately for predicting characteristics after cell culture.

### BRIEF DESCRIPTION OF DRAWINGS

FIG. 1 is a block diagram showing a training device according to a first embodiment.

FIG. 2 is an operation flowchart of a data selection unit of the first embodiment.

FIG. 3 is a block diagram showing a training device according to a second embodiment.

FIG. 4 is a block diagram showing a prediction device for cell characteristic according to a third embodiment.

FIG. 5 is an explanatory diagram of a cell culture device according to a fourth embodiment.

FIG. 6 is an explanatory diagram of a cell culture device according to a fifth embodiment.

### DESCRIPTION OF EMBODIMENTS

Hereinafter, embodiments of the present invention will be described with reference to the accompanying drawings.

### EMBODIMENTS

### First Embodiment

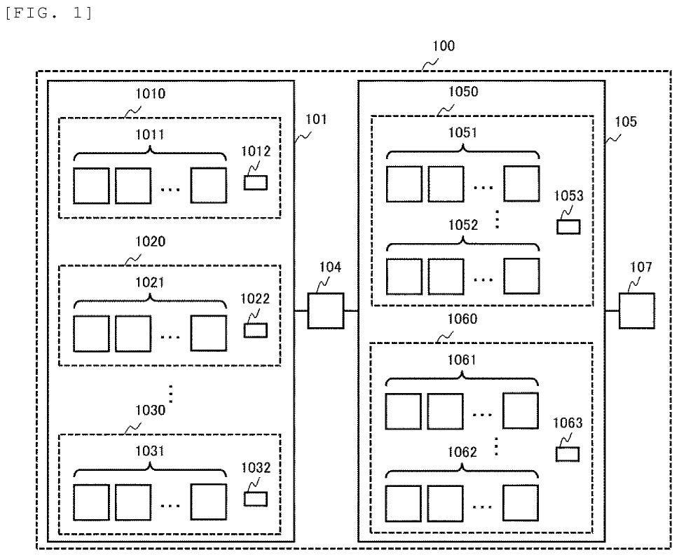

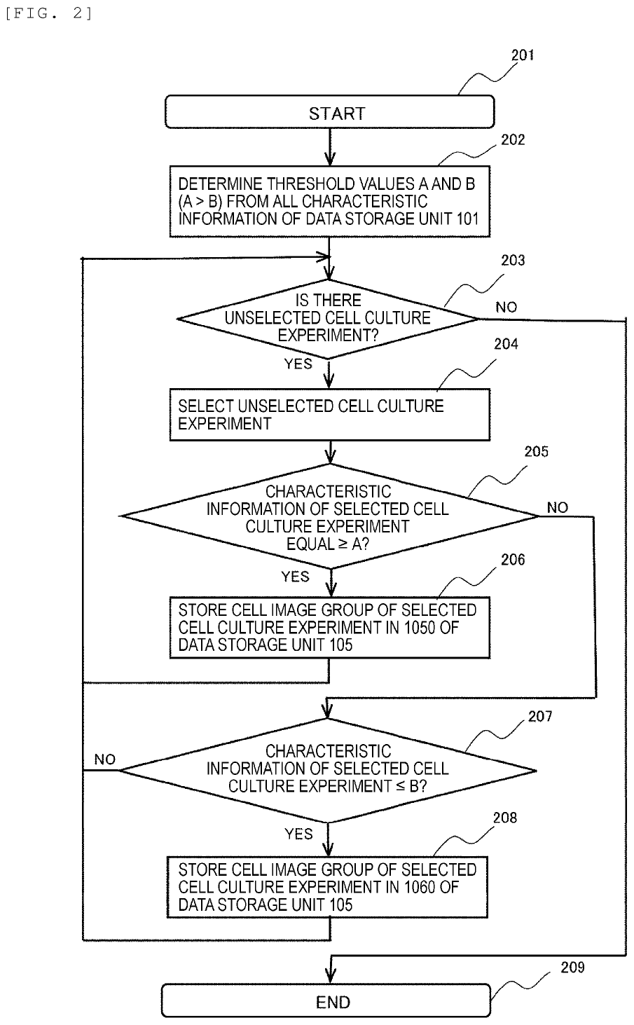

The first embodiment of the present invention will be described using FIGS. 1 and 2.

FIG. 1 is a block diagram showing a training device 100 according to the first embodiment. Referring to FIG. 1, the training device 100 includes a data storage unit 101 (first data storage unit) that stores information acquired from three or more cell culture experiments, a machine learning model 107, a data storage unit 105 that stores machine learning data to be input to the machine learning model 107, and a data selection unit 104 that selects data in the data storage unit 101 and outputs the data to the data storage unit 105 (second data storage unit).

The training device 100 may be a general-purpose computer such as a PC, or a dedicated device. When the training device 100 is a general-purpose computer, the data storage units 101 and 105 are memories or storages.

The acquired information 1010, 1020, . . . 1030 from the cell culture experiment includes a group of cell images 1011, 1021, . . . 1031 during or after culture in each cell culture experiment, and characteristic information 1012, 1022, . . . 1032 after culture. The characteristic information 1012, 1022, . . . 1032 is information indicating characteristics of the cells after culture, such as differentiation induction efficiency, and is information including information indicating a magnitude relationship.

The information indicating the characteristics of the cells after culture may be numerical information indicating a ratio, or information indicating a degree such as large, medium and small. In any case, the information allows the order of characteristic information to be determined.

The data selection unit 104 refers to the characteristic information 1012, 1022, . . . 1032 stored in the data storage unit 101 and d the characteristic information of the cell culture experiments other than the above-mentioned information included in the data storage unit 101, selects a group of cell images excluding a group of cell images of the cell culture experiment having intermediate characteristic information, stores, in the data storage unit 105, a group of cell images of the cell culture experiment having the characteristic information greater than the intermediate characteristic information as machine learning data 1050 corresponding to a label 1053 corresponding to the characteristic information, and stores a group of cell images of the cell culture experiment having the characteristic information smaller than the intermediate characteristic information as machine learning data 1060 corresponding to a label 1063 corresponding to the characteristic information. The machine learning data 1050 includes a group of cell images 1051 . . . 1052, and the machine learning data 1060 includes a group of cell images 1061 . . . 1062.

The data selection unit 104 may be implemented using hardware, software, or the same operation may be performed manually.

FIG. 2 shows an example in which the data selection unit 104 is implemented using software. FIG. 2 is an operation flowchart showing an example of the data selection unit 104.

In FIG. 2, when the process starts (step 201), first, threshold values A and B satisfying A>B are determined from the characteristic information of all cell culture experiments stored in the data storage unit 101 (step 202). At step 202, it is assumed that the characteristic information is a numerical value.

Next, it is determined whether there is unselected cell culture experiment (step 203). At step 203, if there is no unselected cell culture experiment, the process ends (step 209), and if there is an unselected cell culture experiment, the process proceeds to step 204.

At step 204, the unselected cell culture experiment is selected. Then, it is determined whether the characteristic information of the selected cell culture experiment is equal to or greater than A (step 205).

At step 205, if the characteristic information is equal to or greater than A, a group of cell images of the cell culture experiment is stored in the group of cell images 1051 . . . 1052 of the machine learning data 1050 in the data storage unit 105 (step 206), and the process returns to step 203.

If the characteristic information is less than A at step 205, it is determined whether the characteristic information of the selected cell culture experiment is less than or equal to B (step 207).

At step 207, if the characteristic information is less than or equal to B, the group of cell images of the cell culture experiment are stored in the group of cell images 1061 . . . 1062 of the machine learning data 1060 of the data storage unit 105 (step 208), and the process returns to step 203. At step 207, if the characteristic information is not less than or equal to B, the process returns to step 203.

The processes from step 204 to step 208 described above are performed on all the acquired information 1010, 1020 . . . 1030 from the cell culture experiment stored in the data storage means 101.

In the example shown in FIG. 2, it is assumed that the characteristic information after cell culture is a numerical value, but if the characteristic information is qualitative information such as “large”, “medium”, or “small”, a group of cell images of the cell culture experiment with “large” characteristic information may be stored in the machine learning data 1050 of the data storage unit 105, and a group of cell images of the cell culture experiment with “small” characteristic information may be stored in the machine learning data 1060 of the data storage unit 105.

If there are four or more types of qualitative characteristic information, one or more pieces of characteristic information with a continuous magnitude relationship may be determined from the remaining characteristic information excluding the characteristic information corresponding to the maximum the characteristic information corresponding to the minimum, and a group of cell images of the cell culture experiment with larger characteristic information may be stored in the machine learning data 1050 of the data storage unit 105 and a group of cell images of the cell culture experiment with smaller characteristic information may be stored in the machine learning data 1060 of the data storage unit 105.

The machine learning model 107 is trained by using data in which the label 1053 is attached as a training label to some or all of the group of cell images of the machine learning data 1050 in the data storage unit 105, and data in which the label 1063 is attached as a training label to some or all of the group of cell images of the machine learning data 1060 in the data storage unit 105.

It is considered that the cell images from the cell culture experiment with the intermediate characteristic information include more characteristic information from the cell culture experiment with small characteristic information than the cell images from the cell culture experiment with large characteristic information.

It is considered that the cell images from the cell culture experiment with the intermediate characteristic information include more characteristic information from the cell culture experiment with large characteristic information than the cell images from the cell culture experiment with small characteristic information.

Therefore, as described above, by not including the cell images from the cell culture experiment with intermediate characteristic information in the training data, it is possible to reduce the chances of incorrectly labeled data being added to the training data. As a result, the possibility of achieving a more appropriate machine learning model can be increased.

Using the machine learning model trained by the training method or training device described above, by training to infer cell images acquired from the cell culture experiment, select the label corresponding to the cell image with high degree of certainty output by the machine learning model described above, and classify into two classes using some or all of the labels corresponding to the cell images with high degree of certainty, the chances of incorrectly labeled data being added to the training data can be further reduced. As a result, the possibility of achieving a more appropriate machine learning model can be increased.

In other words, according to the first embodiment of the present invention, it is possible to provide a training method and device for a machine learning model that can be trained more appropriately for predicting characteristics after cell culture.

The training method and device according to the first embodiment of the present invention are a training method and device for a machine learning model, including a first step of performing a cell culture experiment at least three times, in which a plurality of cell images are captured during or after culture, and after the cell culture, a plurality of pieces of characteristic information the cells in the plurality of cell images are acquired, and a second step of using some or all of pairs combining the cell image obtained by capturing and the characteristic information of the corresponding cells as training data, in which the second step classifies information indicating a magnitude relationship when arranged in ascending order according to information indicating a magnitude relationship of the characteristic information of the plurality of cell images obtained from the cell culture experiment, into at least three classes including large, medium and small classes, and trains the cell images of the large and small classes as training data. Rather than the cell culture experiment with the maximum characteristic information or the cell culture experiment with the minimum characteristic information, it is also possible to classify the cell culture experiments excluding at least one cell culture experiment with continuous characteristic information into two classes, that is, a class of all the cell culture experiments having characteristic information larger than the characteristic information of the excluded cell culture experiment described above and a class of all the cell culture experiments having characteristic information smaller than the characteristic information of the excluded cell culture experiments described above, and train the classified cell images from the cell experiments as the training data.

The training method and device according to the first embodiment are a training method and device for a machine learning model, in which a cell culture experiment is conducted at least three times, in which a plurality of cell images are captured during or after culture and characteristic information is acquired after culture, and some or all of the obtained pairs of the cell image and the characteristic information are used as the training data, and when specific threshold values A and B (A>B) are determined and the maximum value of the characteristic information is C and the minimum value is D, two classes may be classified for training, that is, a class of all the cell experiments with the characteristic information equal to or greater than min (A, C), and a class of all the cell experiments with the characteristic information less than or equal to max (B, D). In other words, the cell images may be classified into two classes including a class of all the cell images with the characteristic information ranging from the threshold value A to the maximum value C, and a class of all the cell images with the characteristic information ranging from the threshold value B to the minimum value D, and the cell images of the two classes may be trained as the training data.

### Second Embodiment

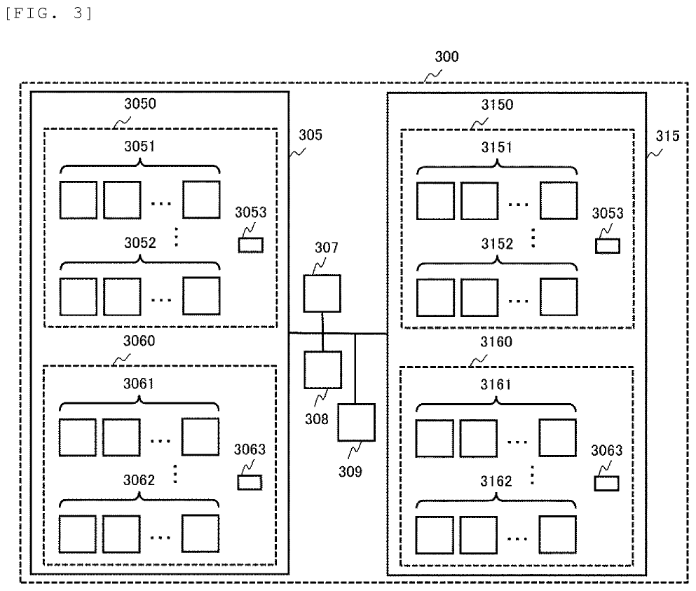

Next, the second embodiment of the present invention will be described using FIG. 3.

FIG. 3 is a block diagram showing a training device 300 according to the second embodiment. Referring to FIG. 3, the training device 300 includes a machine learning model 307, data storage units 305 (third data storage unit) and 315 (fourth data storage unit) that store data for machine learning to be input to the machine learning model 307, a data storage unit 309 (fifth data storage unit) that stores inference results of the machine learning model 307, and a data selection unit 308 that selects a group of cell images in the data storage unit 305 based on the inference result of the data storage unit 309 and stores the result in the data storage unit 315.

The training device 300 may be a general-purpose computer such as a PC, or a dedicated device. When the training device 300 is a general-purpose computer, the data storage units 305 and 315 are memories or storages.

Like the data storage unit 105, the data storage unit 305 stores machine learning data 3050 corresponding to a label 3053 corresponding to the characteristic information and data 3060 corresponding to a label 3063 corresponding to the characteristic information. The machine learning data 3050 includes a group of cell images 3051 . . . 3052, and the data 3060 includes a group of cell images 3061 . . . 3062.

The machine learning model 307 is trained by using, as training data, data in which the label 3053 corresponding to the characteristic information is attached as a training label to some or all of the group of cell images 3051 . . . 3052 of the data 3050 in the data storage unit 305, and data in which the label 3063 corresponding to the characteristic information is attached as a training label to some or all of the group of cell images 3061 . . . 3062 of the data 3060 in the data storage unit 305.

Next, the trained machine learning model 307 infers each of all the groups of cell images 3051 . . . 3052, and 3061 . . . 3062 in the data storage unit 305, and obtains degree of certainty regarding the correct label for each cell image. The inference result is stored in the data storage unit 309.

The data selection unit 308 selects cell images with high degree of certainty regarding the correct label from each of the cell images stored in the data storage unit 305 based on the inference result stored in the data storage unit 309, and stores the cell images in the data storage unit 315 as data corresponding to the correct label.

The data selection unit 308 may be implemented using hardware, software, or the same operation may be performed manually.

The machine learning model 307 is trained by using data in which the label 3053 is attached as a training label to some or all of the group of cell images 3151 . . . 3152 of machine learning data 3150 in the data storage unit 315, and data in which the label 3063 is attached as a training label to some or all of the group of cell images 3161 . . . 3162 of machine learning data 3160 in the data storage unit 315.

In the second embodiment described above, an example using one machine learning model is illustrated, but it is also possible to generate a plurality of machine learning models trained by changing the training data, parameters, initial values of weights that are internal parameters, and the like, comprehensively determine the inference results, and select the data to be stored in the data storage unit 315.

As described above, according to the second embodiment, it is configured to infer all the groups of cell images 3051 . . . 3052, and 3061 . . . 3062 stored in the data storage unit 305, select a group of cell images with a high degree of certainty based on the inference results, and perform training using data with training labels from the selected group of cell images.

Therefore, it is possible to provide a training method and device for a machine learning model, which are capable of training more appropriately for predicting characteristics after cell culture.

The training device 300 according to the second embodiment of the present invention can work separately from the training device 100 according to the first embodiment. The training device 300 according to the second embodiment of the present invention can work together with the training device 100 according to the first embodiment.

### Third Embodiment

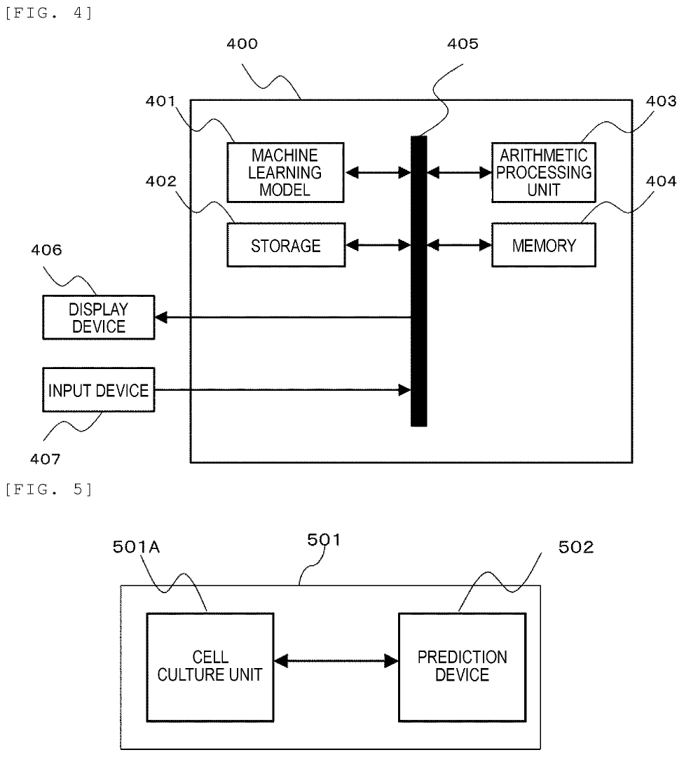

Next, the third embodiment of the present invention will be described using FIG. 4.

FIG. 4 is a block diagram showing a prediction device 400 for cell characteristic according to the third embodiment.

Referring to FIG. 4, the prediction device 400 includes a machine learning model 401, a storage 402, an arithmetic processing unit 403 such as a CPU or GPU, a memory 404, and an internal bus 405.

The prediction device 400 can input and output data using an external display device 406 and an input device 407, and can execute a process such as prediction of cell characteristics. The machine learning model 401 is a machine learning model trained using the training method according to the first embodiment or the second embodiment of the present invention. Therefore, a detailed explanation of the machine learning model 401 will be omitted.

The procedure for predicting cell characteristics is shown below.

First, a group of cell images during or after cell culture is stored in the storage 402. Next, the group of cell images is inferred using the machine learning model 401, the arithmetic processing unit 403, and the memory 404, and the inference result is stored in the storage 402. The arithmetic processing unit 403 refers to the inference result stored in the storage 402 and predicts characteristic information of the group of cell images. Alternatively, an operator (not shown) uses the input device 407 and the display device 406 to refer to the inference result stored in the storage 402 and predicts the characteristic information of the group of cell images.

For example, if the group of cell images includes 10 images, in which 8 cell images are inferred to have “large” characteristic information and 2 cell images are inferred to have “small”, the characteristic information after culture from the cell culture experiment corresponding to the group of cell images is predicted to be “large”.

The prediction device 400 according to the third embodiment includes the storage 402 that stores a plurality of cell images captured during or after cell culture in a cell culture experiment, and a plurality of pieces of inferred characteristic information of the cell images, the machine learning model 401 trained by using the plurality of cell images stored in the storage 402 as training data, the arithmetic processing unit 403 that infers a plurality of pieces of characteristic information of a plurality of cell images stored in the storage 402, and the memory 404 for the arithmetic processing unit 403 to perform calculations, and the like. The arithmetic processing unit 403 predicts the characteristic information of the cells after the cell culture experiment based on the plurality of pieces of characteristic information stored in the storage 402 or the memory 404.

According to the third embodiment, it is possible to provide a prediction device including a machine learning model capable of performing appropriate training for predicting characteristics after cell culture.

### Fourth Embodiment

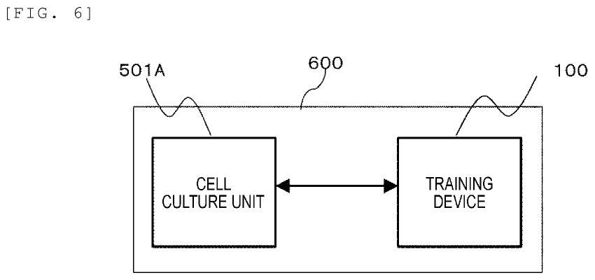

Next, the fourth embodiment of the present invention will be described using FIG. 5.

FIG. 5 is an explanatory diagram of a cell culture device 501 according to the fourth embodiment. In FIG. 5, the cell culture device 501 includes a prediction device 502 having the same configuration as the prediction device 400 according to the third embodiment, and a cell culture unit 501A. The cell culture unit 501A and the prediction device 502 exchange information with each other. Like the prediction device 400, the prediction device 502 includes a machine learning model on which appropriate training can be performed.

The cell culture unit 501A acquires a group of cell images during culture using a camera (not shown) and transmits the group of cell images to the prediction device 502. The prediction device 502 predicts characteristic information after culture.

If the prediction result of the prediction device 502 is good, the operator lets the cell culture unit 501A to continue cell culture. If the prediction result of the prediction device 502 is not good, the operator stops cell culture in the cell culture unit 501A, or takes measures to improve the culture.

According to the fourth embodiment, it is possible to provide a cell culture device that is equipped with the prediction device 502 that can perform appropriate predictions regarding the characteristics prediction after cell culture, and that can determine whether cell culture is good.

### Fifth Embodiment

Next, the fifth embodiment of the present invention will be described using FIG. 6.

FIG. 6 is an explanatory diagram of a cell culture device 600 according to the fifth embodiment. In FIG. 6, the cell culture device 600 includes the training device 100 of the first embodiment and the cell culture unit 501A of the fourth embodiment. The cell culture unit 501A and the training device 100 exchange information with each other.

According to the fifth embodiment, it is possible to provide a cell culture device including a training device of a machine learning model that can be trained more appropriately for predicting characteristics after cell culture.

Note that in the first to fifth embodiments described above, examples in which the cell images are classified into three classes have been illustrated, but the present invention is also applicable to examples in which the cell images are classified into four or more classes. For example, when classifying into four classes, the cell images are classified into large, first medium, second medium, and small. In the training stage, for example, it is preferable to train cell images of large and small classes as the training data. In another example, the “large/first medium” class may be regarded as a group, and the cell images of the class in the group and the cell images of the small class may be used for training. Alternatively, training may be performed using cell images of large and “second medium/small” classes.

In other words, by “training the cell images of the large and small classes as training data”, it may mean that, in a situation where “a class with the largest characteristic information among a plurality of classified classes, or a continuous group of classes including the largest class is regarded as a ‘large class’, and a class with the smallest characteristic information among the plurality of classified classes, or a continuous group of classes including the smallest class is regarded as a ‘small class’, there is at least one class that does not belong to either the large or small class group between the class that forms the ‘large class’ and the class that forms the ‘small class’”, it can be seen that “the cell images of the ‘large class’ and ‘small class’ are trained as training data”.

When five or more classes are classified, it is preferable to classify into large, first medium, second medium, third medium, and small classes, and train, as training data, the cell images of large and small classes, or the cell images of “large/first medium” and small classes, or the cell images of “large/first medium, second medium” and of small classes, or the cell images of “large/first medium” and “third medium/small” classes, or the cell images of large and “second medium/third medium/small” classes, or the cell images of large and “third medium/small” classes.

### REFERENCE SIGNS LIST

100, 300: training device
101, 105, 309: data storage unit
104: data selection unit
107, 307, 401: machine learning model
305, 315: data storage unit
308: data selection unit
400, 502: prediction device
402: storage
403: arithmetic processing unit
404: memory
405: internal bus
406: display device
407: input device
501, 600: cell culture device
501A: cell culture unit
1010, 1020, 1030: information acquired from cell culture experiment
1011, 1021, 1031, 1051, 1052, 1061, 1062, 3051,
3052, 3061, 3062, 3151, 3152, 3161, 3162: group of cell images
1012, 1022, 1032: characteristic information
1050, 1060, 3050, 3060, 3150, 3160: data for machine learning
1053, 1063, 3053, 3063: label corresponding to characteristic information

1. A training method for a machine learning model, the method comprising:
a first step of performing a cell culture experiment at least three times, in which a plurality of cell images are captured during or after cell culture, and after the cell culture, a plurality of pieces of characteristic information of cells of the plurality of cell images are acquired; and
a second step of using some or all of pairs combining the plurality of captured cell images and the characteristic information as training data, wherein
in the second step, information indicating a magnitude relationship of the plurality of pieces of characteristic information of the plurality of cell images is classified into at least three classes including large, medium and small classes, and the cell images of the large and small classes are trained as the training data.

2. The training method according to claim 1, wherein
in the second step, when specific threshold values A and B (A>B) are determined, and a maximum value of the characteristic information is C and a minimum value is D, the cell images are classified into two classes including a class of all the cell images with the characteristic information ranging from the threshold value A to the maximum value C, and a class of all the cell images with the characteristic information ranging from the threshold value B to the minimum value D, and some of the cell images of the two classes are trained as the training data.

3. The training method according to claim 1, wherein the cell images trained as the training data are inferred, and the cell images with a high degree of certainty are further trained as training data.

4. A training device comprising:
a first data storage unit that stores a plurality of cell images captured during or after cell culture and characteristic information of cells of the plurality of cell images;
a data selection unit that classifies information indicating a magnitude relationship of a plurality of pieces of the characteristic information stored in the first data storage unit into at least three classes including large, medium and small classes, and selects the cell images of the large and small classes;
a second data storage unit that stores the cell images of the large and small classes selected by the data selection unit together with the characteristic information; and
a machine learning model trained by using, as training data, some of the cell images of the large and small classes stored in the second data storage unit.

5. The training device according to claim 4, wherein, when specific threshold values A and B (A>B) are determined, and a maximum value of the characteristic information is C and a minimum value is D, the data selection unit classifies the cell images into two classes including a class of all the cell images with the characteristic information ranging from the threshold value A to the maximum value C, and a class of all the cell images with the characteristic information ranging from the threshold value B to the minimum value D, and the cell images of the two classes are the cell images of the large and small classes.

6. A training device comprising:
a third data storage unit that classifies information indicating a magnitude relationship of a plurality of cell images captured during or after cell culture and characteristic information of cells of the plurality of cell images into at least three classes including large, medium and small classes, and stores the cell images of the large and small classes together with the characteristic information;
a machine learning model trained by using, as training data, the plurality of cell images of the large and small classes stored in the third data storage unit, that infers the plurality of cell images and obtains degree of certainty for each of the plurality of cell images;
a fifth data storage unit that stores degree of certainty for each of the plurality of cell images obtained by the machine learning model;
a data selection unit that selects the cell images with the high degree of certainty stored in the fifth data storage unit; and
a fourth storage unit that stores the cell images selected by the data selection unit together with the characteristic information, wherein
the machine learning model is further trained by using, as training data, the cell images stored in the fourth storage unit.

7. A machine learning model trained by using, as training data, the cell images stored in the fourth storage unit according to claim 6.

8. A prediction device comprising:
a storage that stores a plurality of cell images captured during or after cell culture in a cell culture experiment, and a plurality of pieces of characteristic information of inferred cell images;
a machine learning model trained by using, as training data, the plurality of cell images stored in the storage;
an arithmetic processing unit that infers a plurality of pieces of characteristic information of the plurality of cell images stored in the storage; and
a memory for the arithmetic processing unit to perform operations, wherein
the arithmetic processing unit predicts characteristic information of the cells after the cell culture experiment based on the plurality of pieces of characteristic information stored in the storage or the memory.

9. A cell culture device comprising the training device according to claim 4 and a cell culture unit.

10. A cell culture device comprising the prediction device according to claim 8 and a cell culture unit.
